(19) United States
(12) Patent Application Publication
(10) Pub. No.: US 2023/0022119 A1
(43) Pub. Date: Jan. 26, 2023

(54) ABDOMINAL CLOSURE METHOD AND DEVICE VARIATIONS FOR CLOSING VENTRAL HERNIAS AND REDUCING RECURRENCE

(71) Applicant: Absolutions Med. Inc., Mountain View, CA (US)

(72) Inventors: Gregory LAMPS, Smyrna, GA (US); Daniel JACOBS, Mountain View, CA (US)

(21) Appl. No.: 17/805,179

(22) Filed: Jun. 2, 2022

Related U.S. Application Data
(60) Provisional application No. 63/203,429, filed on Jul. 22, 2021.
(60) Provisional application No. 63/260,436, filed on Aug. 19, 2021.

(51) Int. Cl. A61B 17/064

(52) U.S. Cl. CPC A61B 17/0643; A61B 2017/0641

(57) ABSTRACT

Abdominal closure method and device variations for closing ventral hernias and reducing recurrence are described. The tissue anchoring assembly may generally include a first prong defining a lumen at least partially therethrough. The first prong may further define one or more openings or channels at least partially along the first prong and an opening near or at a base of the first prong such that the one or more openings or channels and the opening are in communication via the lumen. A first member may extend from the base of the first prong for contact against a first tissue surface and a second member may be configured to move relative to the first prong for contact against a second tissue surface such that the first member and the second member are secured relative to one another.

### CROSS-REFERENCE TO RELATED APPLICATIONS

This application claims the benefit of priority to U.S. Prov. Apps. 63/203,429 filed Jul. 22, 2021 and 63/260,436 filed Aug. 19, 2021, each of which is incorporated herein by reference in its entirety.

### FIELD OF THE INVENTION

The present invention relates to devices and methods to distribute tension in soft-tissue closure.

### BACKGROUND OF THE INVENTION

There are 4 million open abdominal surgical procedures in the US (emedicine.medscape.com/article/1961789-technique). A commonly documented postoperative complication is incisional hernia resulting from structural failure of healing tissue, which occurs in approximately 9-20% of patients after an abdominal closure, and possibly higher for select risk groups. New technologies and methodologies are desired to reduce the failure rate of abdominal wall closure both primarily and for hernia repair.

### SUMMARY OF THE INVENTION

A device may maintain apposition of the abdominal wall edges during healing by:
a. broadly distributing suture tension throughout the involved tissues,
b. gradually transferring forces from the anchoring device to the healing scar as the device biodegrades, and
c. ideally leaving no permanent foreign body.

One form of the embodiment used for tissue edge apposition includes bioabsorbable components that support tissue closure and optimize healing. For the bioabsorbable components, mechanisms are utilized to form a connection between the posterior (interior of abdominal wall) and anterior (exterior of abdominal wall) elements. These mechanisms may include various fastening mechanisms, e.g., threaded features, notches, ratchet features, threaded features, etc. used in conjunction with securement mechanisms, e.g., ratchets. The mechanisms which form the connection may be reinforced or replaced by suture or similar constructs passed through the posterior and anterior components on both sides of the wound. In some cases, there is no securement mechanism other than suture or similar construct.

In one variation of a tissue anchoring assembly, the assembly may generally comprise a first member having one or more first piercing elements extending from a first surface configured for contact against a first tissue region and a second member having one or more openings corresponding to a position of the one or more first piercing elements, and wherein the first member and second member are configured to be secured relative to one another via the one or more first piercing elements. The one or more piercing elements may be hollow, allowing suture or other connecting member to be passed between the first and second members through the lumen of the piercing element(s). The lumen may have one or more lateral openings through the sides of the piercing elements.

In another variation of a tissue anchoring assembly, the assembly may generally comprise a first prong defining a lumen at least partially therethrough, the first prong further defining one or more openings at least partially along the first prong and a secondary opening near or at a base of the first prong such that the one or more openings and the secondary opening are in communication via the lumen. The assembly may further include a first member extending from the base of the first prong and configured for contact against a first tissue surface, and a second member configured to move relative to the first prong and further configured for contact against a second tissue surface such that the first member and the second member are maintained relative to one another.

In one method of approximating tissue via a tissue anchoring assembly, the method may generally comprise attaching a first tissue anchoring assembly to a first region of tissue (e.g., one side of the abdominal wall incision) and a second tissue anchoring assembly to a second region of tissue (e.g., the second side of the abdominal incision), and approximating the first assembly and region of tissue towards the second assembly and region of tissue by passing a suture through the piercing element of one assembly, through the second assembly, and using the suture to secure the two assemblies such that the two regions of tissue (two sides of the abdominal incision) are in contact.

In another method of approximating tissue via a tissue anchoring assembly, the method may generally comprise piercing a first prong through a tissue region to be approximated such that the first prong extends from a first tissue surface to a second tissue surface of the tissue region, positioning a first member extending from a base of the first prong into contact against the first tissue surface, positioning a second member relative to the first prong and into contact against the second tissue surface opposite to the first tissue surface, and passing a connecting element through a lumen defined at least partially through the first prong and along the second member such that the connecting element passes between the first tissue surface and the second tissue surface.

Examples of different embodiments of the tissue anchoring assemblies and materials which may be used with the features described herein are further disclosed in U.S. Pub. 2020/0323614 (U.S. patent application Ser. No. 16/844,420) filed Apr. 9, 2020 which is incorporated herein by reference in its entirety and for any purpose.

### BRIEF DESCRIPTION OF THE DRAWINGS

FIG. 1A shows a perspective view of an anchor (100) with a single prong (104) and two tines (112) having barbs (116).

FIG. 1B shows a perspective view of an anchor with first (100) and second (120) members with a single prong (104), and one or more tines (112), where the second member can slide along the prong. The second member has a groove (124) for accommodating a connecting element.

FIG. 2A show a perspective view of an anchor with first (100) and second (120) members with a single prong (104), and one or more tines (112), where the second member has fingers (140) which engage with features (142) on the prong and resist movement of the second member along the prong. FIGS. 2B and 2C show perspective views of the anchor with first and second members illustrating the suture trough (124) defined upon the surface and extending from the edge of the second member in alignment with the opening (108) along the single prong.

FIGS. 3A and 3B show cross-sectional side views of an anchor with first (100) and second (120) members illustrating how a suture length (152) or other connecting element may be positioned along the suture trough (124) to extend through the single prong (104) and out along an opening (110) defined along the base (102) of the single prong.

FIG. 4 shows a cross-sectional view of an anchor such as in FIG. 3 used with a similar anchor to approximate tissue (160) in conjunction with a suture (152).

FIG. 5 shows a perspective view of an anchor with first (100) and second (120) members with a single prong (104), and one or more tines (112) without barbs, where the second member has fingers (140) which engage with features (142) on the prong and resist movement of the second member along the prong. The base (102) of the first member is shown having fenestrations (170) and the base (122) of the second member is shown having an additional (third) eyelet (126) in the area of the suture trough (124).

FIG. 6 shows a perspective view of an anchor with first (100) and second (120) members with a single prong (104), and one or more tines (112) without barbs, where the second member has fingers (140) which engage with features (142) on the prong and resist movement of the second member along the prong. The base (102) of the first member is shown having fenestrations (170) and four tines (112) while the second member has eyelets (126).

FIG. 7 shows a perspective view of an alternate design an anchor similar to FIG. 6.

FIG. 8 shows a perspective view of an anchor design consisting of a posterior first member (100) with barbed (116) tines (112), a tri-lobe post (104), and bosses (182) to limit the travel, limiting approximation of the anterior second member (120) towards the posterior first member (100) as it slides on the posterior member post (104).

FIG. 9 shows a cross-sectional view of an anchor where the anterior member (120) has a projection (190) which fits within the slot (108) on the post (104) of the posterior member (100).

FIG. 10 shows a cross-section view of an anchor where the post (104) of the posterior member (100) and the hole (128) of the anterior member (120) are tri-lobed.

FIG. 11A shows a perspective view of an anchor with a penta-lobe post (104), bosses (182) to limit the travel of the second member (120) along the post of the first member (100), tines (112) with barbs (116) on both the first and second members, a reinforcing ring (180) protruding above the surface of the second member around the post hole (128), a protrusion (190) on the hole of the second member which is aligned in the slot (108) on the post of the first member, and concave edge elements (130) on the second member to guide the placement of additional connecting elements which do not directly interact with the first or second members.

FIG. 11B shows a cross-sectional view (through the prong, perpendicular to the platforms) of the anchor of FIG. 11A.

FIG. 11C shows a cross-sectional view (through the platform of the second member, perpendicular to the axis of the prong) of the anchor of FIG. 11A.

FIG. 12 shows a pair of anchors as shown in FIGS. 11A, 11B, and 11C with a connecting element (152) securing the two anterior members (120) relative to the posterior members (100) and the two anchors relative to each other. Not shown is the tissue between the members. The suture path demonstrates curved or radiused surfaces to minimize sharp bends in the suture which may cause high focal stress on the suture leading to possible failure of the suture.

### DETAILED DESCRIPTION OF THE INVENTION

A tissue anchor is described that attaches to soft tissue edges (or near the edges) and allows for the application of distributed tensile force across said soft tissues.

The embodiments for the soft tissue anchor described may be made partially or entirely from bioabsorbable (bioresorbable) materials. Bioabsorbable materials may include, but are not limited to, polylactic acid (PLA), polyglycolic acid (PGA), lactic/glycolic acid copolymers (PLGA), polydioxanone (PDO, PDS), trimethylene carbonate (TMC), and polycaprolactone (PCL). Also included in this bioresorbable materials are the various copolymers of these materials such as PLA-co-TMC or PLGA-co-PDO.

Alternatively, the devices may be made partially or entirely from materials that are not bioabsorbable. Non-bioabsorbable materials could include stainless steel, titanium, polyethylene (PE), polypropylene (PP), polyetheretherketone (PEEK), polyphenylene sulfide (PPS), or other materials which do not significantly degrade in the body.

The various embodiments may have antibiotics, active pharmaceuticals, and/or antimicrobials as a surface coating or incorporated into the material.

The prongs and tines described below may have any of a variety of geometries. These include, but are not limited to, cylindrical shaped, oval, rectilinear, conic, parabolic, or other shapes. The tips of the prongs and tines (104, 114) can also take multiple forms such a cutting, atraumatic, traumatic, multi-bevel, ‘pencil’ tip, etc. The prong may have a protrusion or change in diameter that limits the extent to which the second member can glide along the post towards the first member. In addition, the tines and prongs may be barbed or have other features which tend to resist any tendency of the tine to pull out of the tissue. The prong(s) are hollow or partially hollow forming a lumen along at least part of the length of prong. In addition the prong has one or more openings along the length of the prong which allows a connecting element such as suture which is passed through the lumen of a prong to exit at desired point(s) or along a desired region of the prong.

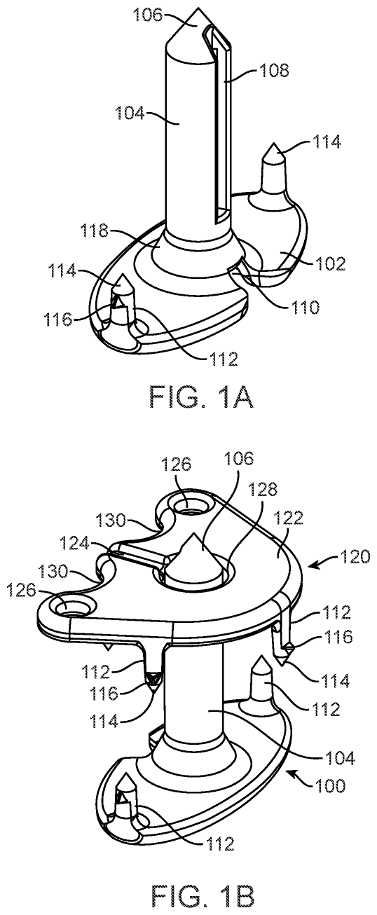

In one embodiment (perspective view of FIG. 1A), an anchor (100), having a base or platform (102), has one or more prongs (104) which penetrate through the abdominal wall including the peritoneum (optionally), the posterior rectus sheath (fascia, to the extent that it is present), the muscles, and anterior rectus sheath (fascia) where tensile strength is greatest, and/or tines (112) intended to penetrate only a partial thickness of the abdominal wall. The anchor may comprise a base defining one or more openings therethrough with one or more prongs that may project away from the base either in a normal direction or angled relative to a plane defined by the base. The prong(s) may also define a first diameter or thickness, e.g., 1 to 6 mm, and may also define any number of cross-sectional shapes, including round, oval, triangular, square, or other polygon whether regular or not. Additionally, the prongs may extend at a distance of, e.g., 7 to 25 mm (although longer lengths may be needed for unusually thick abdominal walls), to help ensure that the prongs can extend through the entire thickness of the abdominal wall to which the anchor is to be secured. Excess portions of the lengths may be removed as described in further detail herein. One or more of the prongs are hollow, presenting a lumen (150) of sufficient length such that all or substantially all of the prong length in the tissue is hollow. The lumen is such a diameter that one or more pieces of a surgical suture can be passed through the lumen (lumen preferably 0.5 mm diameter or larger). The lumen may also have a non-circular shape. One section of the wall of such prong may have one or more openings (108) such that the prong may be truncated after insertion into the tissue and allow the suture to exit the lumen without being elevated above the level of the surrounding tissue by the wall of the prong. One or more tines, shorter than the prong(s), may be arranged on the base and configured such that they pass completely or partially through the tissues of the abdominal wall. The tines and/or prongs may have undercuts or barbs (116) which would tend to resist the device disengaging from the tissues.

The prongs may also be positioned over the base to provide a stable securement relative to the tissue to which they are attached. The example shown illustrates one prong (104) extending from the center of the base (102) but other variations may utilize greater numbers of prongs which may be positioned at alternate locations of the base. The prongs may have undercuts or barbs which would tend to resist the device disengaging from the tissues.

Tines (112) may also be incorporated to extend from the base such that they each define a second diameter or thickness, e.g., 1 to 6 mm, and may also define any number of cross-sectional shapes, as described hereinabove. Additionally, the tines may extend at a relatively shorter distance of, e.g., 1 to 8 mm, than the prongs to help ensure further engagement with the tissue but may not extend through the entire thickness of the tissue. Furthermore, the tines may be positioned at multiple positions on the base around the prong or around and in-between the prongs if more than one prong is present. The tines may have undercuts or barbs (116) which would tend to resist the device disengaging from the tissues.

While the one or more tines (112) may be used to pierce partially into the tissue and help secure a position of the base, the one or more tines may be optionally omitted entirely from the base (102). Alternatively, other variations may include a base having the one or more tines, each configured with a length commensurate with a length of the prong such that the one or more tines extend entirely through the tissue thickness. The lengths of the one or more tines extending from the tissue surface may be left unaltered or they may be optionally truncated so that the one or more tines are reduced in length so as to remain flush with the tissue surface.

The variations of the tines being omitted entirely or varied in length, for example, extending entirely through the tissue thickness, may be combined in any combination with any of the different embodiments described herein.

To protect the viscera, the anchors are typically installed with the prongs passed from the inside of the abdomen towards the outside. This safety measure does not restrict the anchors to only that usage and an alternative is to pass the prong from the outside of the abdomen towards the inside.

The anchor may be composed of two members, the first (100) as shown in FIG. 1A paired with a second member (120) configured to be used in conjunction with the first member in order two sandwich a region of tissue between the two members with the prong(s) (104) of the first member passing through the tissue and through hole(s) (128) on the second member such that the second member can be moved along the prong(s) of the first member. The second member consisting of a base (122) which may optionally have one or more tines (112) configured on the surface of the base which is in contact with tissue. The tine(s) preferably have a tip (114) configured to penetrate tissue and may optionally have one or more barbs (116) arranged along the length of the tine(s). The second member may also have one or more eyelets (126) configure on the platform to allow for a connecting element such as suture to be passed through the eyelet(s). The platform of the second member may also have a depression such as a groove or trench (124) along the surface of the platform which is configured to extend from a periphery of the platform to the hole (128) such that the groove or trench (124) allows the connecting element to lie in close proximity to the surface of the tissue. The groove or trench (124) may also prevent the connecting element from sliding over the surface of the platform. The groove is positioned such that it aligns from a location at or near the hole for the prong in the platform towards an edge of the platform. In the case where the prong has hole(s) (108) in the side to allow the connecting element to exit along the length of the prong lumen, the groove in the second platform is ideally aligned with the hole in the prong.

With a matching anchor on the opposite side of the wound or incision, a connecting element which may be a flexible and biocompatible element such as a suture (152) can then be passed through the lumens and connect the suture anchors on each side of the incision to one another, thus holding the edges of the abdominal wall in apposition during healing. In another embodiment, the components may be reversed such that the base or platform may be inserted from the exterior with prongs through the wall. The associated internal base of the anchor in the abdomen may then have a thinner section or trough that allows the suture passing on the internal surface of the abdominal wall to rest on, within, or only slightly proud of the posterior surface of the abdominal wall thereby minimizing the risks to the viscera.

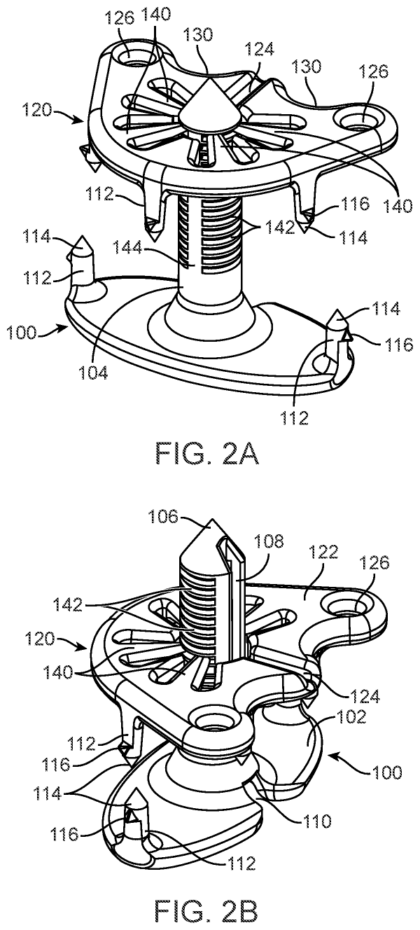

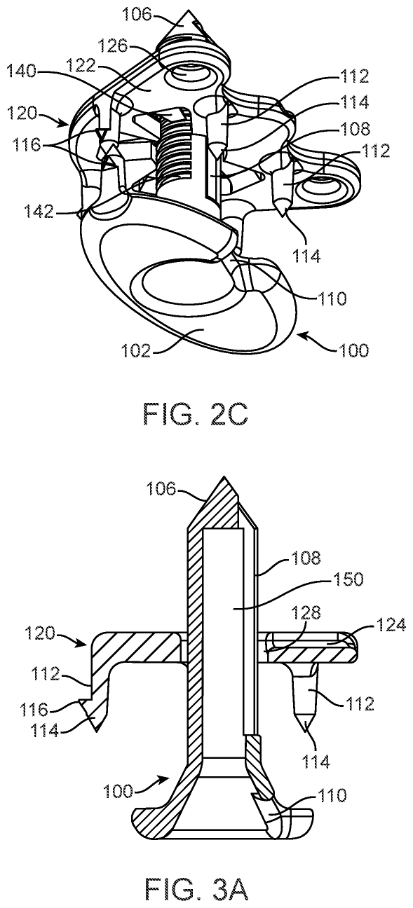

Anchors similar to the previous embodiments may be coupled to a second member (120) having a base or platform (122) (as shown in the perspective views of FIG. 2), preferably superficial to the anterior rectus sheath (fascia). In such embodiments the anchor may comprise a base (first member) (102) with one or more prongs (104) that are placed from the interior of the abdominal wall to its external surface, with the prongs extending through the thickness of the wall and a base or platform (second member) (122) on the exterior of the wall which clips, snaps, screws, or is otherwise attached to the prong(s) from the first member (100). The variation shown in FIG. 2 illustrates how the second member (120) may also comprise a base defining one or more openings (128) and one or more tines (112) which extend from the base of the second member in the same manner as tines (112) of the first member (100). In some embodiments the second member is able to slide on the prong(s) of the first member. In such a case, a suture or other connecting element can be used to hold the first member and second member in position such that the prong(s) does not slide out of the receiving structures of the second member. It is also desirable in some embodiments to arrange one or more structures which limit the sliding of the first and second member relative to each other. Features such as snaps (190) near the distal tips of the prong(s) can assist maintaining the second member on the prong of the first member prior to suture placement or prevent the second member from sliding off the first member. Bosses or other features such as the tines may be arranged and dimensioned on one or both of the platforms or on the post(s) to limit how closely together the platforms may slide. In the embodiment shown in FIG. 8, protrusions (182) are arranged at one or more locations around the prong of a first member, limiting how close the platform of the second member can be to the platform of the first member.

As similarly described with respect to the one or more tines (112) extending from the base (first member) (102), the one or more tines (112) extending from the second member (120) may be used to pierce partially into the tissue and help secure it. In one variation, the one or more tines may be optionally omitted entirely from the second member. Alternatively, other variations may include a second member having the one or more tines each configured with a length commensurate with a length of the prong such that the one or more tines extend entirely through the tissue thickness. The lengths of the one or more tines extending from the tissue surface may be left unaltered or they may be optionally truncated so that the one or more tines are reduced in length so as to remain flush with the tissue surface.

The variations of the tines (112) being omitted entirely or varied in length from the second member (120) may be combined in any combination with any of the different embodiments of the one or more tines (112) extending from the base (first member) (102) and are intended to be within the scope of this description. For example, the first member (100) and second member (120) in one variation may both omit the tines entirely. In another variation, the first member may include the one or more tines while the second member may omit the tines entirely or vice versa. In yet another variation, the first member and second member may each include the respective one or more tines each with uniform lengths or each having different lengths from either or both of the first and second members.

One or more of the openings (128) or receiving channels of the second member may further correspond to the positioning of the prong(s) (104) extending from the first member (100). In this manner, the second member (120) may be positioned so that the prong(s) from the first member extends through the corresponding opening(s) defined in the second member while allowing for the first member and the second member to maintain an orientation relative to one another while sandwiching the tissue thickness between each of the members. The one or more tines (112) of the second member may be oriented to point towards the first member, as shown, to further secure the tissue between each of the members.

With a corresponding matching anchor on the opposite side of the wound or incision, a connecting element (152), e.g., wire, suture, etc. can then be passed through the lumens (150) and connect the suture anchors on each side of the incision to one another, thus holding the edges of tissue in apposition during healing and/or reinforcing an additional tissue repair at the incision. In addition, the suture can act to hold the bases of both the first and second members in contact with the tissue. In another embodiment, the components may be reversed such that the base or platform may be inserted from the exterior with prong(s) through the wall. The bases of the anchors may have a thinner sections or troughs (110,124) that allows the suture passing on the surface of the abdominal wall to rest on, within, or only slightly proud of the face of the abdominal wall.

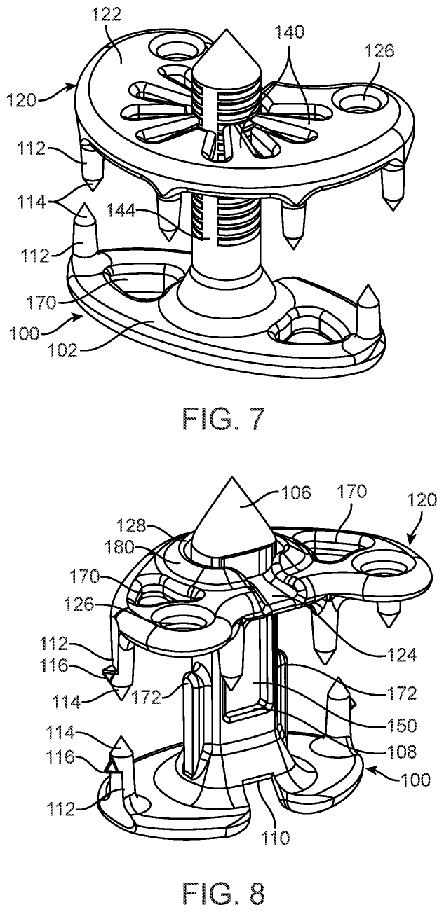

In several embodiments (FIGS. 2 to 8), the anchor comprises a first member (100) (also referred to as a posterior plate or base), with one or more prongs (104) extending perpendicularly from the plate (102), as previously described, that are placed from the interior of the abdominal wall with the prongs extending through the thickness of the abdominal wall towards the external surface and a second member (120) (also referred to as an anterior plate or base), on the exterior of the abdominal wall, preferably subcutaneously. The second member may have integrated features such as one or more flexible finger(s) (140) that engage with one or more notch(es) (142) or similar geometry on the prongs from the plate of the first member. The flexible fingers may be configured as cantilevered members which are biased into contact against the prong surface to allow the second member to slide down the prongs towards the first member but may or may not have a different force resisting sliding off (away from the plate) due to engagement of the fingers against the notches when urged in the opposite direction. The fingers or pawls may be arranged on one or more sides of the prongs. In FIGS. 2A-2C, flexible fingers are shown which act on multiple sides of the prongs. Specifically, a posterior member has a prong or prongs with ridges or rings provided on all or part of each prong. In the example of fingers on the second member (FIGS. 2A, 2B, and 2C), the fingers (140) engage the notch (142) on the prong to hinder separation from the prong. The example shown has four fingers but the number of fingers can be varied based on the diameter of the prong, the force to resist motion relative to the prong, or limitation of manufacturing methods. Similarly the thickness, length, and width of the fingers can be varied to achieve desired forces and accommodate the materials and methods of construction. In some embodiments the fingers and notches may be configured such that they can only be engaged in one rotational orientation around the prong or so that after engagement of the fingers to the notches that the ability of the second member to rotate around the axis of the prong is similarly limited. In FIG. 2A this is achieved with one or more longitudinal ribs (144) interrupting the circumferential notches (142) around the prong.

FIGS. 2A, 2B, and 2C show perspective views of the anchor with the first and second members to illustrate a connecting channel (124) such as a groove or trough for receiving the connecting element such as a suture where the connecting channel is defined upon the surface of the second member (120) and extending from the edge of the platform (122) of the second member in alignment with the opening (108) along the single prong. In addition, the first member illustrates a groove or slit (110) on the plate that allows the connecting element to lie flush or sub-flush with the tissue surface.

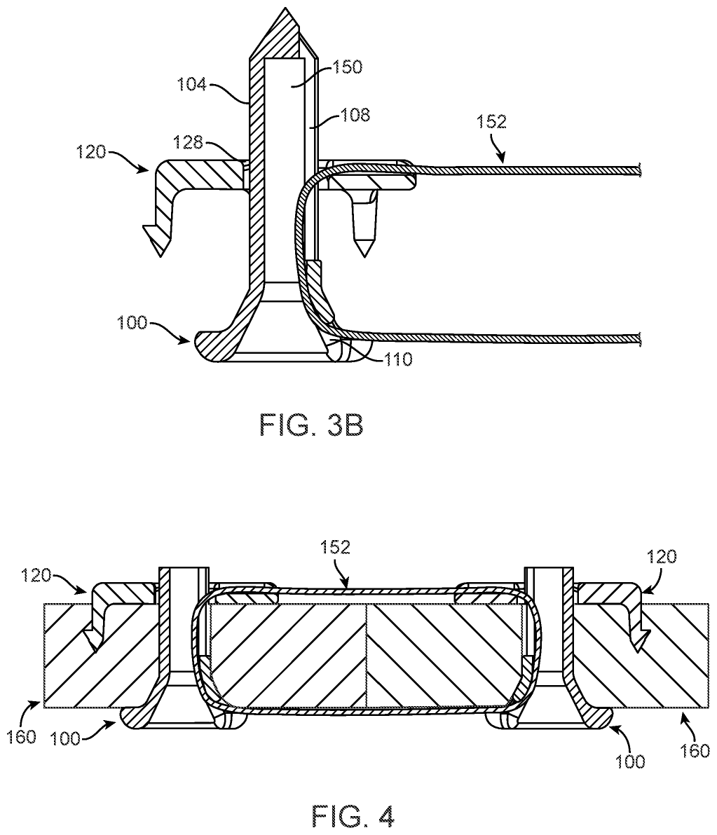

FIGS. 3A and 3B show cross-sectional side views of the anchor with the first and second members illustrating how a suture length (152) may be positioned to lie within the channel of the second member (120) along the platform (124) of second member to extend through one or more openings (108) or channels in communication with the lumen (150) defined at least partially through a length of the single prong (104). As shown, the suture may extend through the lumen and out along a secondary opening (110) defined along the base of the single prong or along the first member. With the first and second members secured to, for example, the internal and external surfaces of the tissue, the suture may reside and slide, if needed, along the connecting channel of the second member, within the lumen, and along the opening of the single prong without directly contacting the tissue so that tearing of the suture through the tissue is reduced.

As shown in FIG. 4, with a matching anchor on the opposite side of the wound or incision, a suture (152) can then be passed through the lumens (150) and connect the suture anchors on each side of the incision to one another, thus holding the edges of tissue (160) in apposition during healing and/or reinforcing an additional tissue repair at the incision. In addition, the suture can act to hold the bases (102,122) of both the first and second members (100,120) in contact with the tissue, thus lessening the strength required in, for example, flexible fingers to hold the members in position relative to each other. In another embodiment, the components may be reversed such that the base or platform may be inserted from the exterior with prongs through the wall. The bases of the anchors may have a thinner sections (110,124) or troughs that allows the suture passing within the surface of the abdominal wall to rest on, within, or only slightly proud of the surface of the abdominal wall.

Similar to the other embodiments described, a first member (100) having one or more posts (104) with lumen(s) and optionally one or more tines (112) and a slot (110) through the base for the connecting element to lay on the surface or slightly in the surface can be combined with a second member (120) with one or more holes (128) corresponding to the one or more posts of the first members and further optionally having one or more tines (112). In this embodiment, the second member has a reinforcing ring (180) around the post hole(s) which may be raised. Such ring(s) may optionally have projection(s) (190) which fit into slot(s) (108) on the post(s). Additionally, in some cases it is desirable to radius the base of the post(s), and the surfaces of the ring(s) and projection(s) so that the connecting element (152) such as suture has a smooth path without sharp corners or small radii which would otherwise cause a localized stress concentration in the connecting element(s) and potentially lead to premature failure of the connecting elements. Both the first and/or second members bases may have one or more edge contours (130) to indicate to the user where to place additional suture or other reinforcing elements that while not passing through the described plates are located in some proximity to the members to help maintain the wound edges in apposition.

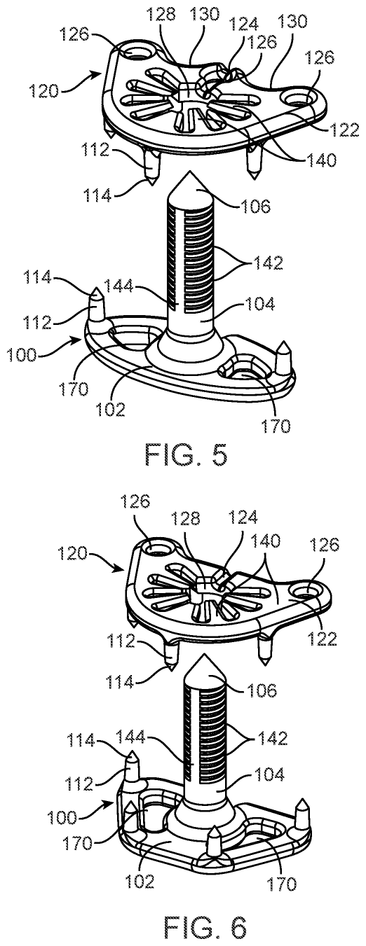

FIGS. 5, 6, and 7 show a variety of embodiments demonstrating different configurations that are possible. These include adding fenestrations (170) to the first or second member to reduce mass, tailor the strength, or encourage tissue ingrowth. The number and shapes of the tines (112) on both members can also be tailored to change the area of device which penetrates the tissue and thereby distributes the forces acting on the anchor across a larger or smaller area of tissue contact. In contradistinction to FIGS. 1 and 2 which include barbes (116) on some or all of the tines of both members, FIGS. 4, 5, and 6 show tines with no barbs. The number and locations of eyelets (126) on the members can also be changed to allow for more or less engagement points for suture to interact with the anchors. The shape and sizes of the members can also encompass a range of shapes including circular, semi-circular, triangular, quadrilateral, or any desired shape to enable the desired device performance elements including numbers of eyelets, tines, fenestrations and ratchet fingers. Any of the first members (or similar shapes) can be combined with any of the second members (or similar shapes).

In each of these embodiments described, the first member (100) may alternatively be referred to as a posterior plate or base due to its relative positioning within the body and the second member (120) may alternatively be referred to as an anterior plate or base also due to its relative positioning within the body and relative to its position with respect to the posterior plate or base.

In embodiments where any of the anchors described above and the suture are bioabsorbable, each of these components may bioabsorb into the patient's body, thus avoiding a permanent footprint of foreign material that can later lead to complications such as infection.

One or more holes (126) may be placed in either or both members of the device to allow for suture passage, as shown in anterior plates (122) in the various embodiments herein. The suture from one anchor can connect to an anchor on the contralateral side in a running pattern so that each of the anchors is interconnected to the other. In other examples, one anchor may be attached to another anchor via suture in an interrupted pattern forming a pair of anchors. The suture and anchor complex may hold the abdominal wall edges in apposition while healing occurs and/or reinforcing an additional tissue repair at the incision. The suture holes may be provided on the anterior, posterior, or multiple members of the anchor.

In many embodiments described, the prong(s) (104) extending from the first member (100) may be cut, broken, or melted above the second member (120) after assembly to limit excessive length of prongs protruding above the second member into or through subcutaneous tissues superficial to the abdominal wall.

In several of the embodiments described, such as in the case of only one prong (104) extending from a first member (100) through a second member (120), it is advantageous to include features to control the angle of the second member around the axis of the prong of the first member. These features may include one or more projections (190) from one of the members arranged to fit into a slot (108) of the other member. Alternatively, the same functionality may be achieved by using a non-round, non-uniform profile on the prong and the mating hole in the second member.

FIG. 8 shows a tri-lobe prong (104) which mates with a tri-lobe hole (128) in the second member (120). This prong is hollow for the majority of its length with a slot (108) extending from the base of the tip (106) to a fixed distance above the platform (102). The stops (172) on the sides of the prong may be configured to be longer than the tines (112) of both platforms. The hole (128) of the second member is slightly larger than the prong and the stops on the prong extend from the sides of the prong an overlapping dimension such that when the second member slides along the prong, the rim of the hole comes into contact with the surface of the stops and limits further motion of the second member towards the platform (102) of the first member (100). The height of the stops above the platform of the first member is configured such that the base of the slot (108) in the wall of the prong does not extend above the upper rim of the hole in the second member. In some cases it may be desirable to eliminate the stops on the prong and instead to utilize the tines contacting the opposing platform(s) to limit the translation of the second member along the prong of the first member. A similar height of the tines relative to the slot position on the prong as described for the stop position is desirable. The stops are configured of a size and shape to resist the forces that are expected to be encountered.

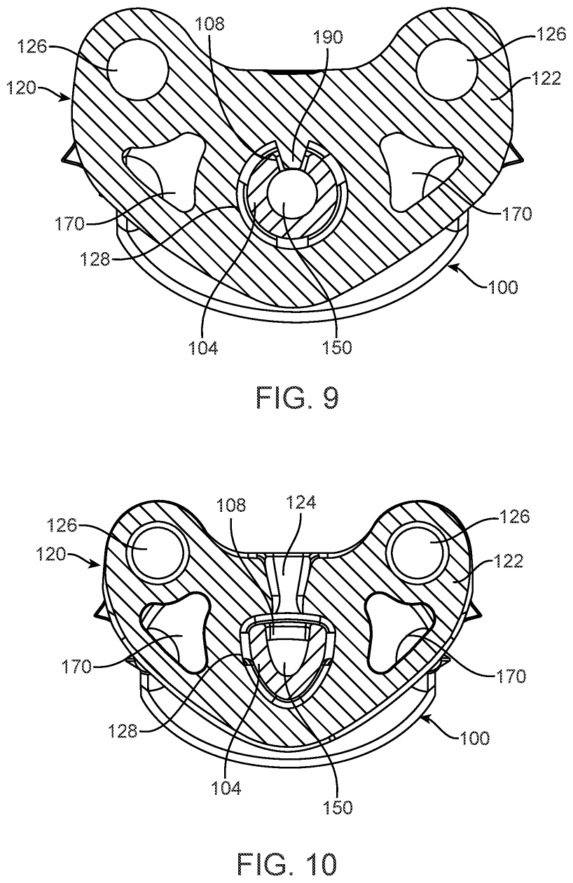

In embodiments with only one prong, it is desirable to limit the rotation of the second member around the axis of the prong of the first member. One embodiment which achieves this goal includes a round prong (104), a round hole (128) in the second member configured to pass along the prong, and a tongue-like projection (190) from the inner edge of the hole that engages with the slot (108) of the prong. This embodiment is shown in FIG. 9 which is a cross-sectional view through the platform of the second member. The projection interacts with the slot to limit rotation of the second member around the longitudinal axis of the prong. The surfaces of the projection of the second member can be configured to provide a radiused or otherwise smooth surface to any connecting element passed through the lumen of the anchor and over this projection.

In other embodiments it is desirable to use a non-circular prong (104) on the first member (100) and a similarly shaped hole (128) in the second member (120) to limit rotation of the second member around the longitudinal axis of the first member. This embodiment is shown in FIG. 10 which is a cross-sectional view through the platform of the second member. In this case, the prong is tri-lobal.

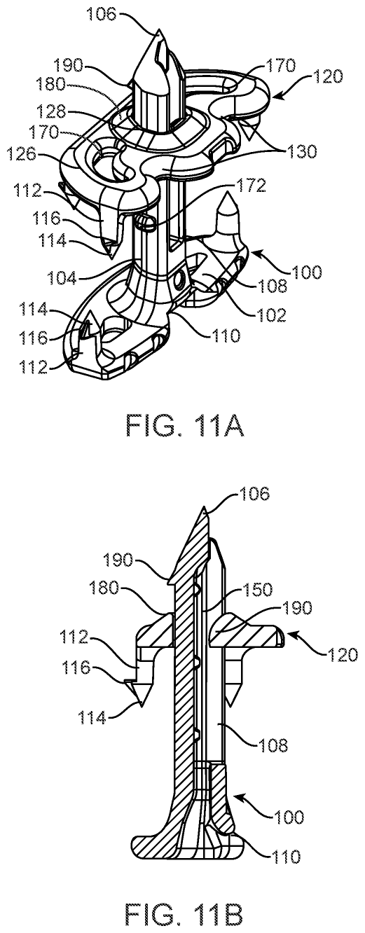

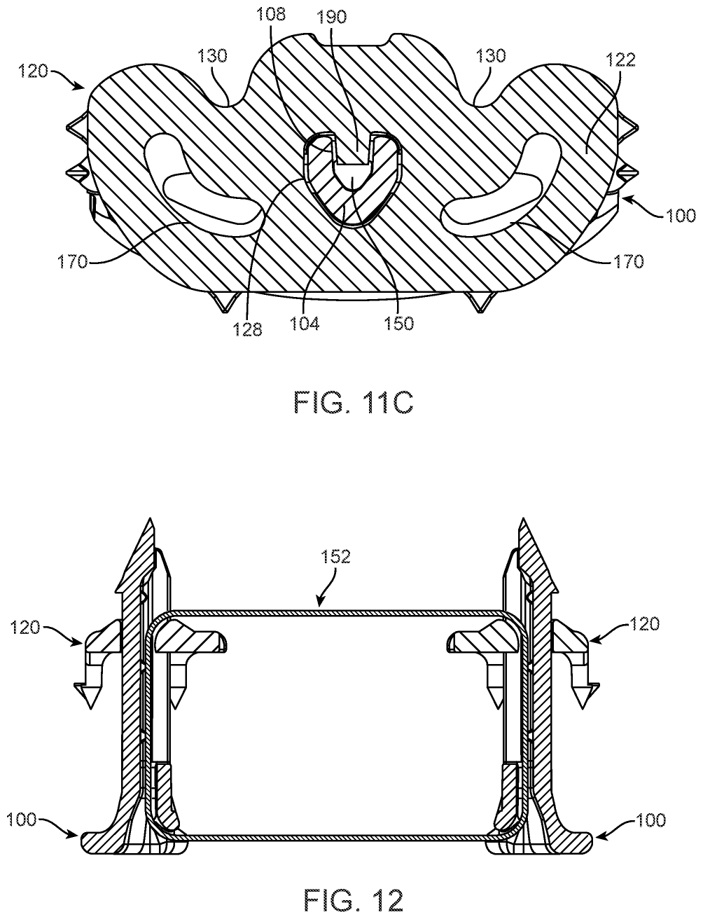

One embodiment combining a number of the features described in previous examples is shown in FIGS. 11A (perspective view), 11B (cross-sectional view through the prong perpendicular to the platforms), and 11C (cross-sectional view through the platform of the second member perpendicular to the prong axis). The first member (100) consists of a platform (102) with one hollow prong extending from the platform. The prong is penta-lobal in cross section with a slot (108) extending from near the tip (106) to a short distance above the platform. Two small stops (172) are arranged on the sides of the prong. Two shorter tines (112) with barbs (116) are arranged on the platform. Additionally, the platform has two fenestrations (170) and a slot (110) which allows a suture to rest against the surface of the tissue that the anchor is in rather than being suspended from the surface by the platform. A second member (120) may define a hole (128) in the second platform (122) with the hole having a similar penta-lobal shape as the prong. A projection (190) from the rim of the hole extends into the slot of the prong. The hole also has a raised rim (180) to increase strength. Similarly, to the first member, the second member also has multiple barbed tines (four) (112). Rather than eyelets for additional suture engagement, two semi-circular notches (130) are arranged on one edge of the second member to provide a visual guide for placing sutures which do not directly engage with the anchor.

The first member (100) of one anchor can be inserted into tissue so that the prong (104) and tines (112) penetrate into the first surface of a region of tissue with the distal end protruding beyond the opposite surface of the tissue. The second member (120) can then be pushed down over the exposed prong such that the tines (112) of the second member penetrate the second surface of the tissue. A second anchor can be inserted in a similar fashion into another region of tissue separate from the first anchor by an incision or gap. The two regions of tissue can then be approximated toward each other by placing a connecting member (152) through the lumens (150) of both anchors and through the holes (128) of the second members. Such a connecting member can then be used to approximate the two regions of tissue toward each other. Multiple such pairs of anchors can be used together to approximate a larger region of tissue.

FIG. 12 shows a pair of anchors as shown in FIGS. 11A, 11B, and 11C with a connecting element (152) securing the two anterior members (120) relative to the posterior members (100) and the two anchors relative to each other. Not shown is the tissue between the members. The suture path demonstrates curved or radiused surfaces to minimize sharp bends in the suture which may cause high focal stress on the suture leading to possible failure of the suture.

It is specifically contemplated that any of the features described with respect to a particular embodiment may be combined with the features of any other embodiment such that any number of combinations are permissible.

The applications of the devices and methods discussed above are not limited to wound closure but may include any number of further treatment applications. Moreover, such devices and methods may be applied to other treatment sites within the body. Modification of the above-described assemblies and methods for carrying out the invention, combinations between different variations as practicable, and variations of aspects of the invention that are obvious to those of skill in the art are intended to be within the scope of the claims.

What is claimed is:

1. A tissue anchoring assembly, comprising:
a first prong defining a lumen at least partially therethrough, the first prong further defining one or more openings at least partially along the first prong and a secondary opening near or at a base of the first prong such that the one or more openings and the secondary opening are in communication via the lumen;
a first member extending from the base of the first prong and configured for contact against a first tissue surface; and
a second member configured to move relative to the first prong and further configured for contact against a second tissue surface such that the second member translates along the first prong relative to the first member.

2. The assembly of claim 1 further comprising one or more first piercing tines extending from a first surface of the first member.

3. The assembly of claim 1 further comprising one or more second piercing tines extending from a second surface of the second member.

4. The assembly of claim 1 wherein the second member defines a connecting channel along a surface of the second member which extends between an edge of the second member and the one or more openings defined along the first prong.

5. The assembly of claim 1 wherein the second member defines one or more openings along an edge of the second member.

6. The assembly of claim 1 wherein the second member defines a receiving opening sized to receive the first prong within.

7. The assembly of claim 1 further comprising a connecting element positionable to extend through the opening near or at the base of the first prong, through the lumen, and through the receiving opening of the second member.

8. The assembly of claim 7 further comprising a second tissue anchoring assembly which is connectable to a first tissue anchoring assembly via the connecting element.

9. The assembly of claim 1 further comprising at least one stop member positioned to inhibit the platform of the second member from advancing towards the platform of the first member beyond the at least one stop member.

10. The assembly of claim 9 wherein the at least one stop member is positioned along the first prong.

11. The assembly of claim 10 further comprising a second stop member positioned along the first prong opposite to the at least one stop member.

12. The assembly of claim 2 wherein the one or more piercing tines comprise an undercut or barb.

13. A method of approximating tissue via a tissue anchoring assembly, comprising:
piercing a first prong through a tissue region to be approximated such that the first prong extends from a first tissue surface to a second tissue surface of the tissue region;
positioning a first member extending from a base of the first prong into contact against the first tissue surface;
positioning a second member relative to the first prong and into contact against the second tissue surface opposite to the first tissue surface; and
passing a connecting element through a lumen defined at least partially through the first prong and along the second member such that the connecting element passes between the first tissue surface and the second tissue surface.

14. The method of claim 13 wherein the connecting element extends through an opening near or at the base of the first prong.

15. The method of claim 13 wherein the connecting element extends along a surface of the second member.

16. The method of claim 13 wherein positioning the first member comprises piercing one or more first piercing tines extending from the base into the first tissue surface.

17. The method of claim 13 wherein moving the second member comprises piercing one or more second piercing tines extending from the second member into the second tissue surface.

18. The method of claim 13 wherein moving the second member comprises adjusting a position of the second member along the first prong such that the tissue region is secured between the first member and the second member.

19. The method of claim 18 wherein the second member defines a receiving opening sized to receive the first prong within.

20. The method of claim 13 wherein passing the connecting element comprises positioning the connecting element within a channel defined along a surface of the second member which extends from an edge of the second member.

21. The method of claim 13 wherein the connecting element passes adjustably between the first tissue surface and the second tissue surface.

22. The method of claim 13 further comprising inhibiting a movement of the second member towards the first member via one or more stop members.

23. The method of claim 13 further comprising securing the connecting element to a second tissue anchoring assembly secured to a second tissue region to be approximated with the tissue region.

24. The method of claim 23 further comprising approximating the tissue region and the second tissue region towards one another by tightening the connecting element.

25. The method of claim 23 further comprising securing the second member to the second tissue anchoring assembly via one or more openings along an edge of the second member.
